(19) United States
(12) Patent Application Publication
(10) Pub. No.: US 2023/0277139 A1
(43) Pub. Date: Sep. 7, 2023

(54) RAPID DIAGNOSIS OF POTENTIAL TRAMATIC BRAIN INJURY OF USERS IN SITU

(71) Applicant: Kyndryl, Inc., New York, NY (US)

(72) Inventors: Caroline K. Cameron, Durham, NC (US); Corville O. Allen, Morrisville, NC (US)

(21) Appl. No.: 17/592,045

(22) Filed: Feb. 3, 2022

(51) Int. Cl. A61B 5/00; G16H 50/20; A61B 5/369; A61B 5/0205

(52) U.S. Cl. CPC A61B 5/7275; A61B 5/0006; A61B 5/0205; A61B 5/369; G16H 50/20; A61B 2503/10

(57) ABSTRACT

Systems and method of the invention provide an in situ diagnostic system for traumatic brain injury (TBI). In implementations, a method includes: receiving, by a computing device, real-time user parameter data from one or more sensors of the user during a monitoring event; writing, by the computing device, the real-time user parameter data as time series data in a data store; determining, by the computing device, that at least one parameter of the real-time user parameter data meets or exceeds a predetermined parameter threshold value; calculating, by the computing device, a diagnostic score for the user based on the time series data, baseline parameter data of the user, and a determined protective equipment profile of the user; and automatically diagnosing a potential traumatic brain injury (TBI) of the user in situ based on the diagnostic score meeting or exceeding a diagnostic threshold.

### BACKGROUND

Aspects of the present invention relate generally to medical monitoring systems and, more particularly, to a system for automatic rapid diagnosis of a potential traumatic brain injury (TBI) of users in situ.

As our medical understanding of the cause and effect of TBI improves, various systems have been developed to detect impacts on a user. For example, various smart helmet systems have been developed for detecting impact to a user’s head. Additionally, screening techniques have been developed for assessing a user post-traumatic event. Further, various wearable systems have been developed to monitor vital signs of the user for various applications.

### SUMMARY

In a first aspect of the invention, there is a computer-implemented method including: receiving, by a computing device, real-time user parameter data from one or more sensors of the user during a monitoring event; writing, by the computing device, the real-time user parameter data as time series data in a data store; determining, by the computing device, that at least one parameter of the real-time user parameter data meets or exceeds a predetermined parameter threshold value; calculating, by the computing device, a diagnostic score for the user based on the time series data, baseline parameter data of the user, and a determined protective equipment profile of the user; and automatically diagnosing a potential loss of consciousness (LOC) and/or a potential traumatic brain injury (TBI) of the user in situ based on the diagnostic score meeting or exceeding a diagnostic threshold.

In another aspect of the invention, there is a computer program product including one or more computer readable storage media having program instructions collectively stored on the one or more computer readable storage media. The program instructions are executable to cause a computing device to: receive real-time user parameter data from one or more sensors of the user during a monitoring event, the real-time user parameter data including physiological parameter data and impact parameter data; write the real-time user parameter data as time series data in a data store; determine that at least one parameter of the real-time user parameter data meets or exceeds a predetermined parameter threshold value; calculate a diagnostic score for the user based on the time series data, baseline parameter data of the user, and a determined protective equipment profile of the user; and automatically diagnose a potential TBI of the user in situ based on the diagnostic score meeting or exceeding a diagnostic threshold.

In another aspect of the invention, there is system including a processor, a computer readable memory, one or more computer readable storage media, and program instructions collectively stored on the one or more computer readable storage media. The program instructions are executable to cause a computing device to: receive real-time user parameter data from one or more sensors of the user during a monitoring event, the real-time user parameter data including physiological parameter data and impact parameter data; write the real-time user parameter data as time series data in a data store; determine that at least one parameter of the real-time user parameter data meets or exceeds a predetermined parameter threshold value; calculate a diagnostic score for the user based on the time series data, baseline parameter data of the user, and a determined protective equipment profile of the user; automatically diagnose a potential TBI of the user in situ based on the diagnostic score meeting or exceeding a diagnostic threshold; and send an alert based on the diagnosis to a remote participant device of a user.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a cloud computing node according to an embodiment of the present invention.

FIG. 2 depicts a cloud computing environment according to an embodiment of the present invention.

FIG. 3 depicts abstraction model layers according to an embodiment of the present invention.

FIG. 4 shows a block diagram of an exemplary environment in accordance with aspects of the invention.

FIG. 5 shows a flowchart of an exemplary method in accordance with aspects of the invention.

FIG. 6 is a diagram representing exemplary use scenarios with different tiers of user protection.

### DETAILED DESCRIPTION

Aspects of the present invention relate generally to medical monitoring systems and, more particularly, to a system for automatic rapid diagnosis (assessment or determination) of a potential traumatic brain injury (TBI) of users in situ (i.e., in the place the monitoring event is occurring). In embodiments, a system is provided for monitoring functional disturbance of a user to detect TBI, including a mild TBI (e.g., concussion).

There have been more than 408,000 TBIs reported in service members since 2000, and an estimated 1.6-3.8 million sports-related TBIs occur every year. However, there is inconsistent reporting of these occurrences due to adrenaline, post-traumatic amnesia, confusion, or other reasons. A loss of consciousness (LOC) and other vital sign changes during head trauma can indicate a potential concussion or TBI. Embodiments of the invention provide a system that can monitor user consciousness during blast exposures and head impacts to detect or diagnose potential loss of consciousness or TBI. In implementations, an algorithm of the system correlates vital sign measurements such as heart rate, electrocardiogram (ECG), blood pressure, blood sugar, respiratory patterns, and body temperature with blast sensor and/or head impact sensor profiles factored with a level of protective equipment of the person. In aspects, the resulting data demonstrates an occurrence of a functional disturbance for concussion and TBI detection analysis and severity determination. Implementations of the invention provide insight on the correlations of protective equipment, blast effect behind armor, and impact on head trauma. Systems of the invention may have multiple applications, including for military use during training or combat, and for athletes during a sporting event.

In embodiments, a system includes a set of sensing devices (e.g., wearable sensing devices) to measure medical or physiological parameters (e.g., vital signs) of a user such as heart rate, ECG records, blood pressure, blood sugar, respiratory rate, and body temperature. In implementations, the information obtained from the sensing devices is stored in a database system, per user, to establish parameter baselines (e.g., low, normal, high) within some standard deviation. These baselines, once established, are stored in the system, per user, as user baselines. In embodiments, the sensing devices write the measures for medical or biological parameters (e.g., vital signs) at specified intervals or based on a triggering event (e.g., a high impact event). In aspects, the system then writes the measurements from all the sensing devices to a datastore in a time series. In implementations, the system utilizes the time series to detect, in real time, a sudden change in one or more baseline measurements (e.g., vital sign measurements), which the system then uses to determine a functional disturbance or predicted LOC.

In embodiments, a shock or impact sensor (e.g., blast sensor) records an impact event. The impact sensor may be in the form of one or more blast sensors (e.g., worn inside a helmet and on a uniform or other wearable gear). In implementations, the system records a blast profile that measures intensity, duration, and overpressure. In embodiments, a prediction of a functional disturbance or LOC is adjusted by the system based on the user’s safety equipment profile or level of protection. In aspects, the system adjusts measured parameters (e.g., vital signs) of a user based on the safety equipment profile or level of protection of the user, and utilizes the adjusted parameters in a scoring algorithm configured to predict a functional disturbance or LOC.

Thus, implementations of the invention provide a system for automatic rapid diagnosis of a potential TBI for a user in-situ. Embodiments of the invention provide for analysis of real-time digital sensor data in a manner of seconds, rather than minutes. Therefore, implementations of the invention provide an improvement over more time consuming manual diagnostic methods by enabling near-immediate diagnosis of a potential injury. Moreover, embodiments of the invention do not require an in-person evaluation of a user, and enable diagnosis of potential injury to users who are currently participating in an event, such as a user at work or a user engaged in a sporting event. Implementations of the invention do not merely recite the performance of a manual diagnostic method using generic computer components. Rather, embodiments of the invention provide a special purpose monitoring device configured to diagnose potential TBI of a user in-situ based on wearable sensing devices of the user.

It should be understood that, to the extent implementations of the invention collect, store, or employ personal information provided by, or obtained from, individuals (for example, vital sign measurements), such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium or media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service’s provider.

Broad network access: capabilities are available over a network and accessed through standard mechanisms that promote use by heterogeneous thin or thick client platforms (e.g., mobile phones, laptops, and PDAs).

Resource pooling: the provider’s computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider’s applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

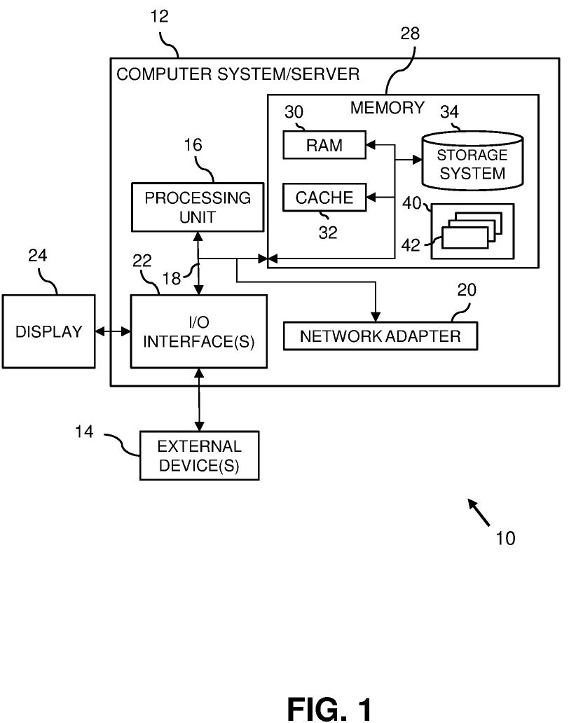

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

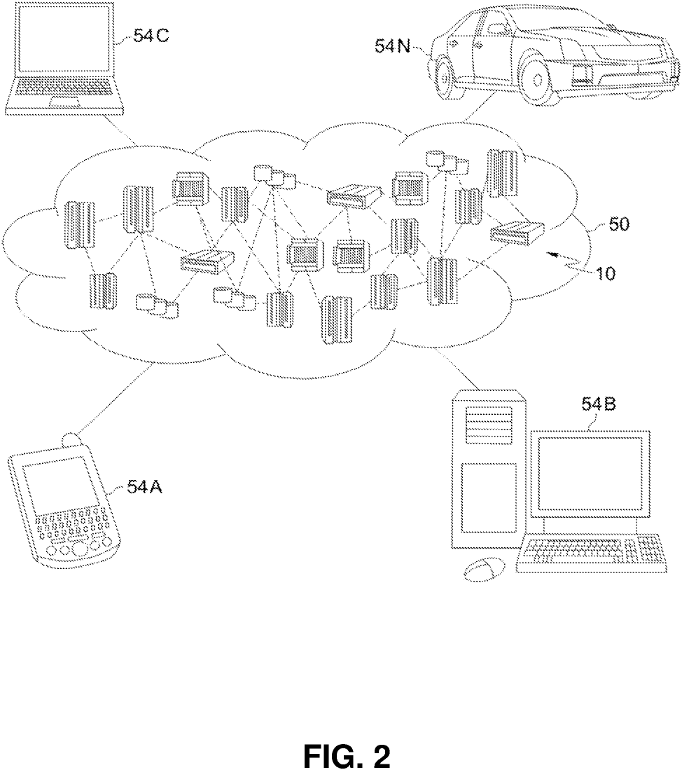

Referring now to FIG. 2, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

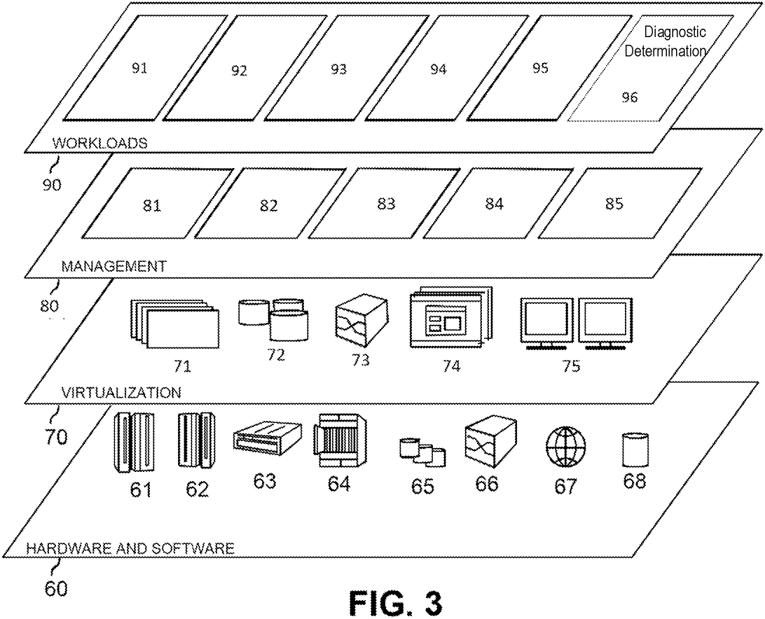

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and diagnostic determination 96.

Implementations of the invention may include a computer system/server 12 of FIG. 1 in which one or more of the program modules 42 are configured to perform (or cause the computer system/server 12 to perform) one of more functions of the diagnostic determination 96 of FIG. 3. For example, the one or more of the program modules 42 may be configured to: receive real-time user parameter data from one or more sensors of the user during a monitoring event, the real-time user parameter data including physiological parameter data and impact parameter data; write the real-time user parameter data as time series data in a data store; determine that at least one parameter of the real-time user parameter data meets or exceeds a predetermined parameter threshold value; calculate a diagnostic score for the user based on the time series data, baseline parameter data of the user, and a determined protective equipment profile of the user; automatically diagnose a potential traumatic brain injury (TBI) of the user in situ based on the diagnostic score meeting or exceeding a diagnostic threshold; and send an alert based on the diagnosis to a remote participant device of a user.

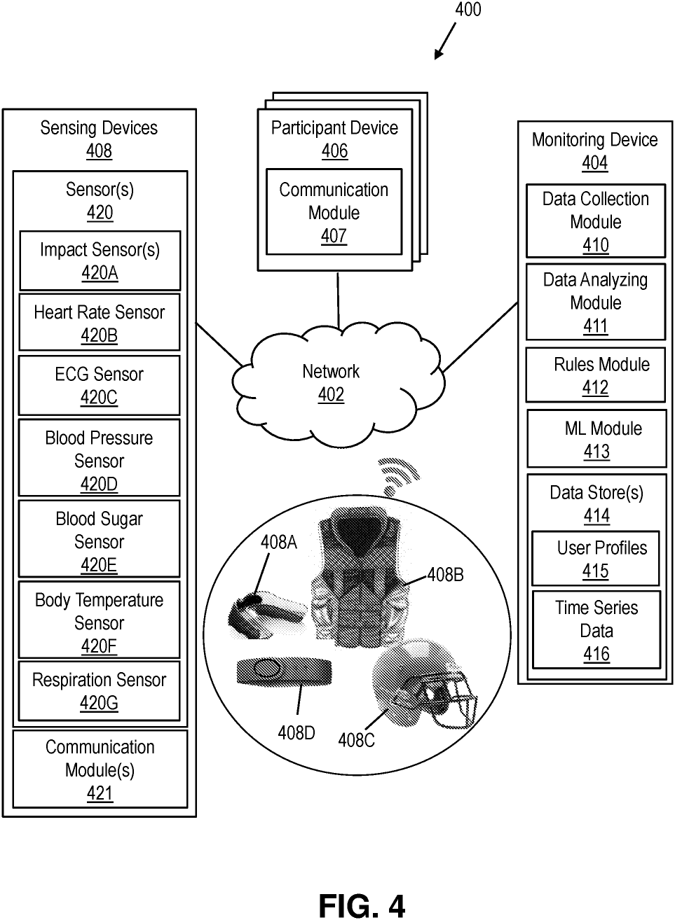

FIG. 4 shows a block diagram of an exemplary environment 400 in accordance with aspects of the invention. In embodiments, the environment 400 includes a network 402 enabling communication between a monitoring device 404, one or more participant devices 406, and one or more sensing devices 408. The monitoring device 404, one or more participant devices 406, and one or more sensing devices 408 may each comprise the computer system/server 12 of FIG. 1, or elements thereof. Additionally, the monitoring device 404, one or more participant devices 406, and one or more sensing devices 408 may be computing nodes 10 in the cloud computing environment 50 of FIG. 2. Various sensing devices 408, such as smart helmet systems, may be utilized in embodiments of the invention, and the type of sensing devices 408 utilized are not intended to be limited to the examples herein.

In embodiments, the monitoring device 404 comprises a cloud-based server providing diagnostic services to one or more users in the environment 400. In implementations, the one or more participant devices 406 comprise local computing devices used by cloud consumers, such as, for example, the personal digital assistant (PDA) or cellular telephone 54A, the desktop computer 54B, and/or the laptop computer 54C of FIG. 2.

In embodiments, the monitoring device 404 comprises one or more modules, each of which may comprise one or more program modules such as program modules 42 described with respect to FIG. 1. In the example of FIG. 4, the monitoring device 404 includes a data collection module 410, a data analyzing module 411, a rules module 412, and a machine learning (ML) module 413. In implementations, the monitoring device 404 also includes one or more data stores 414 for storing digital information, including user profiles 415 and time series data 416.

In embodiments, the data collection module 410 is configured to collect data (e.g., digital data) from one or more sensing devices 408 of a plurality of users. The data may include signal data from one or more sensors 420 providing parameter information (e.g., vital sign and impact information) from the one or more sensing devices 408. Parameters monitored by the monitoring device 404 may include, for example, heart rate; ECG; blood pressure; blood sugar; body temperature; and respiratory rate, as well as impact (e.g., blast impact or direct impact). In aspects, the monitoring device 404 saves parameter data for a user as time series data 416 in the data store 414.

In implementations, the data analyzing module 411 is configured to analyze the collected data from the data collection module 410 to determine baselines for the parameter information (e.g., baselines for vital sign parameters) of individual users and associated standard deviations. The baseline parameter and standard deviation information may be saved in user profiles 415 for each user in the data store 414. Further, in embodiments, the data analyzing module 411 is configured to score an event (e.g., an impact event) based on the time series data 416 and scoring rules to determine if parameter information obtained for a user indicates that a medically significant event has occurred (e.g., a loss of consciousness). In aspects, the data analyzing module 411 is configured to generate and send notifications and/or alerts to one or more sensing devices 408 and/or one or more participant devices 406 (e.g., to a communication module 407 of a mobile computing device).

In embodiments, the rules module 412 is configured to store rules utilized by the data analyzing module 411 to establish baselines and/or perform event scoring. In implementations, the stored rules are configurable by authorized users, and/or are automatically configurable by the monitoring device 404 (e.g., based on ML analysis).

In embodiments, historic user data obtained over time for multiple users is utilized by the ML module 413 to provide insights into how to accurately predict that a medically significant event has occurred for a user based on parameter data obtained for the user. In aspects, the ML module 413 is configured to utilize ML algorithms to classify stored user data and/or detect patterns in stored user data. In implementations, an output of the ML module 413 comprises parameter thresholds and/or scoring values based on historic user data (e.g., parameter monitoring data and medical data obtained by third parties), wherein the parameter thresholds and/or scoring values may be utilized by the monitoring device to determine when a medically significant event (LOC event) has occurred for a particular user. In implementations, the monitoring device 404 is configured to utilize parameter thresholds and/or scoring values in combination with a level of protection of the user (e.g., the user’s protective equipment profile) to determine that an impact event is medically significant (e.g., an LOC event or TBI is likely). In embodiments, a user’s protective equipment profile includes types of protective gear utilized by the user and information thereon (e.g., impact ratings, specification, etc.). Examples of protective gear include, but are not limited to, helmets or other protective headgear, body armor, pads, footwear, mouthguards, clothing and glasses or other eye protection.

In embodiments, each of the one or more sensing devices 408 comprises one or more modules, each of which may comprise one or more program modules such as program modules 42 described with respect to FIG. 1. The one or more sensing devices 408 may comprise internet of things (IoT) devices, may include one or more wearable devices, and may be incorporated into protective gear of a user. Some examples of sensing devices 408 include a mouthguard 408A, a protective vest 408B, a protective helmet 408C and a wrist-mounted sensing device 408D. In the example of FIG. 4, the one or more sensing devices 408 include one or more: impact sensors 420A, heart rate sensors 420B, ECG sensors 420C, blood pressure sensors 420D, blood sugar sensors 420E, body temperature sensors 420F, and respiration sensors 420G.

In implementations, one or more communication modules 421 enable communication between the one or more sensors 420 and the monitoring device 404. It should be understood that each of the one or more sensing devices 408 may include hardware and/or software enabling information to be communicated to another of the sensing devices 408 and/or the data collection module 410 of the monitoring device 404. Communication between the one or more sensing devices 408 and/or the monitoring device 404 may be wireless communication via Bluetooth or other wireless communication methods. For example, a communication module 421 may be incorporated into the protective vest 408B or the protective head gear 408C, to enable wireless communication of parameter information for a user to the data collection module 410 of the monitoring device 404. The one or more communication modules 421 may comprise one or more modules (e.g., program modules 42 described with respect to FIG. 1).

The monitoring device 404, one or more participant devices 406, and one or more sensing devices 408 may include additional or fewer modules than those shown in FIG. 4. In embodiments, separate modules may be integrated into a single module. Additionally, or alternatively, a single module may be implemented as multiple modules. Moreover, the quantity of devices and/or networks in the environment 400 is not limited to what is shown in FIG. 4. In practice, the environment 400 may include additional devices and/or networks; fewer devices and/or networks; different devices and/or networks; or differently arranged devices and/or networks than illustrated in FIG. 4.

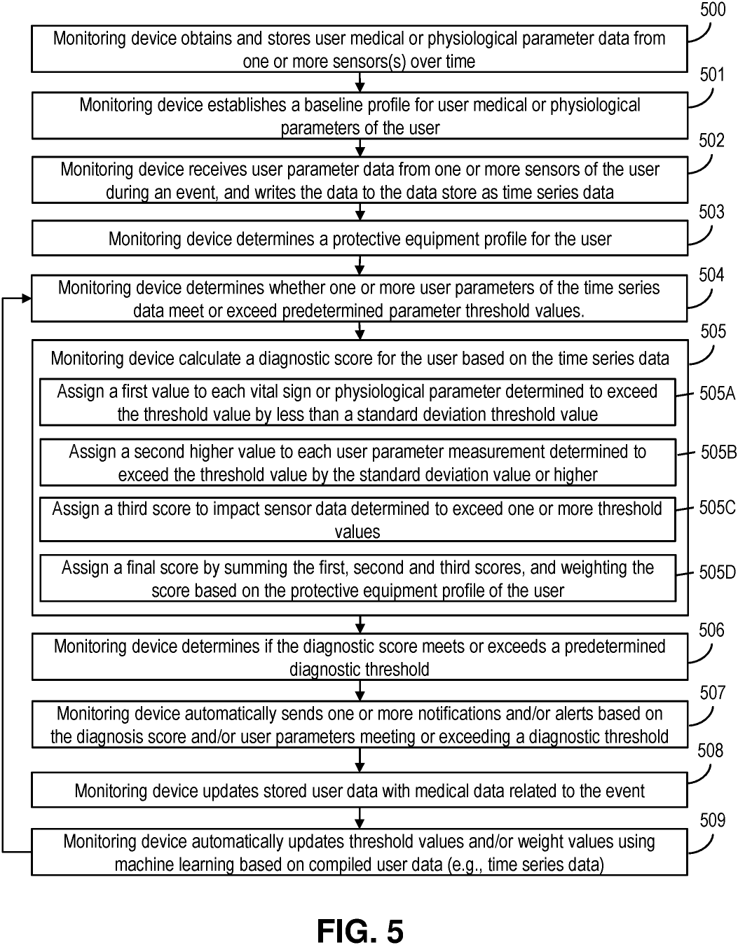

FIG. 5 shows a flowchart of an exemplary method in accordance with aspects of the present invention. Steps of the method may be carried out in the environment of FIG. 4 and are described with reference to elements depicted in FIG. 4.

At step 500, the monitoring device 404 obtains user medical or physiological parameter data (e.g., vital sign data) for a user from one or more sensing devices 408 over time, and saves the data in a data store (e.g., data store 414). In implementations, the medical or physiological parameter data includes measurements for one or more of: heart rate, ECG, blood pressure, blood sugar, body temperature, and respiratory rates or patterns. In implementations, the data collection module 410 obtains the user medical or physiological parameter data from one or more communication modules 421 of one or more of: the heart rate sensor 420B, the ECG sensor 420C, the blood pressure sensor 420D, the blood sugar sensor 420E the body temperature sensor 420F and the respiration sensor 420G of FIG. 4. Alternatively, the data collection module 410 may obtain user medical or physiological parameter data from another source, such as a participant device 406 of the user or a third party. Medical or physiological parameter data from other sensors not discussed herein may be utilized, and the invention is not intended to be limited to the examples provided herein.

At step 501, the monitoring device 404 establishes a baseline profile of user medical or physiological parameters based on the stored user parameter data. The baseline profile of a user may be stored as a user profile 415 in the data store 414. In embodiments, the baseline values for a user are calculated for one or more of the following user parameters: heart rate, ECG, blood pressure, blood sugar, body temperature, and respiratory rates or patterns. In implementations, the baseline profile of a user includes a baseline measurement or value for each user medical or physiological parameter and associated standard deviation values. In implementations, the baseline values comprise a baseline average calculated by summing the measurements or values of the data entries and dividing by the total number of data entries. The term standard deviation value as used herein refers to a quantity calculated to indicate an extent of deviation for a group as a whole (e.g., a measure of how dispersed data is in relation to a mean). In embodiments, the data analyzing module 411 of the monitoring device 404 implements step 501. The table below represents vital sign or physiological parameters of a user in an exemplary user profile in accordance with embodiments of the invention, with standard deviations for the user represented in parenthesis ().

TABLE 1

Exemplary Vital Sign Parameters of a User Profile

| PARAMETER | LOW | NORMAL | PREDIABETIC | HIGH | ABNORMAL |
|---|---|---|---|---|---|
| Heart Rate | < 60 (3) | 60-100 (3) | n/a | >100 (3) | n/a |
| ECG | n/a | 0 | n/a | n/a | 2 |
| Blood Pressure | ≤ 90/60 (3) | 120/80 (0) | n/a | ≤ 130/50 (3) | n/a |
| Blood Sugar | n/a | < 140 (3) | 140-199 (3) | >200 (3) | n/a |
| Body Temperature | <97 (3) | 98.6 (0) | n/a | >100.4 (3) | n/a |

At step 502, the monitoring device 404 receives user parameter data (e.g., medical, or physiological parameter data and impact data) from one or more sensing devices 408 of the user during an event, and writes the user parameter data to a data store 414 as time series data 416. In general, time series data is a collection of observations for a subject at different time intervals, wherein data points are indexed in time order. The term event as used herein refers to monitoring event of a certain duration (e.g., during a sporting event, within a certain time period, etc.), during which the one or more sensing devices 408 (e.g., wearable sensors) are configured and arranged to obtain user parameter data of the user in situ. An event may be associated with a predetermined time period, the duration of some event, or a period of time that a user is wearing or otherwise engaged with sensing devices 408 (e.g., wearing protective gear). In implementations, the monitoring device 404 receives user parameter data continuously, or at predetermined intervals during the event. Alternatively, the monitoring device 404 may receive user parameter data based on a triggering event occurring. A triggering event may comprise one or more of the sensing devices 408 sensing a predetermined triggering condition (e.g., an impact sensor 420A measures an impact over a threshold value). In embodiments, the data collection module 410 of the monitoring device 404 implements step 502.

Table 2 illustrates thresholds associated with an impact sensor on a protective helmet. In one example, a triggering event is determined to occur when impact parameters from an impact sensor 420A indicate a direct trauma with a high force of impact and a high acceleration of the head.

TABLE 2

Exemplary Impact Threshold Values

| IMPACT PARAMETERS | VALUE | HIGH | MODERATE | LOW |
|---|---|---|---|---|
| Direct Trauma | 3 | n/a | n/a | n/a |
| Indirect Trauma | 2 | n/a | n/a | n/a |
| Force of Impact | n/a | 3 | 2 | 1 |
| Acceleration of Head | n/a | 3 | 2 | 1 |

Table 3 illustrates thresholds associated with a blast sensor on body armor. In one example, a triggering event is determined to occur when blast parameters from an impact sensor 420A indicate a high peak of an initial pressure wave and a high duration of overpressure.

TABLE 3

Exemplary Blast Threshold Values

| BLAST PARAMETERS | HIGH | MODERATE | MINIMAL |
|---|---|---|---|
| Peak of Initial Pressure Wave | 3 | 2 | 1 |
| Duration of Overpressure | 3 | 2 | 1 |

At step 503, the monitoring device 404 determines a protective equipment profile for the user. In embodiments, the protective equipment profile comprises a predetermined type or level of protection from a plurality of predetermined types or levels of protection. A type of protection may be associated with a particular group of protective equipment (e.g., American football equipment, soccer equipment, military, or law enforcement gear, etc.). In implementations, the monitoring device 404 obtains the protective equipment profile of a user from the user (e.g., during registration) via a user interface provided by the monitoring device 404 (e.g., the user may select one of the predetermined protective equipment profiles). Alternatively, the monitoring device 404 may obtain information (e.g., make, model, capabilities/specifications, etc.) regarding protective equipment of a user from the protective equipment itself (e.g., from an IoT helmet) or from another device, and may determine the protective equipment profile of the user based on that information and predetermined profile rules. In embodiments, the protective equipment profile of a user is stored as part of the user profile 415 in the data store 414. In embodiments, the data analyzing module 411 of the monitoring device 404 implements step 503.

At step 504, the monitoring device 404 determines whether one or more user parameters of the time series data meet or exceed predetermined thresholds, in real time. In implementations, the monitoring device 404 accesses the user’s baseline profile, and analyzes the time series data in real time to determine if each user medical or physiological parameter in the time series data exceeds the user’s established baseline value for that parameter by a predetermined threshold amount. In embodiments, the monitoring device 404 determines if one or more of the following parameters in the time series data exceeds predetermine threshold values for those parameters based on the user’s baseline profile: heart rate, ECG, blood pressure, blood sugar, body temperature, and respiratory rate or pattern. For example, the monitoring device 404 may compare incoming heart rate data for the user to the established baseline heart rate from the user’s user profile 415, and may determine if the incoming heart rate data has a value that is higher than the baseline heart rate by more than a predetermined (threshold) amount based on threshold values in the rules module 412.

In aspects, the predetermined threshold amount for one or more user parameters varies depending on the protective equipment profile of the user. For example, a threshold value for an impact parameter may be different for a profile indicating a higher level of protection than for a profile indicating a lower level of protection. In implementations, the monitoring device 404 makes the determination of step 504 after detecting a triggering event, such as a sudden change in a parameter value (e.g., a change within a predetermined period of time over a threshold value). In embodiments, the monitoring device 404 determines if impact parameters (e.g., from a helmet impact sensor or blast sensor) meet or exceed predetermined impact threshold values. In implementations, the impact threshold value is determined based on the user’s protective equipment profile. In embodiments, the data analyzing module 411 of the monitoring device 404 implements step 504.

At step 505, the monitoring device 404 calculates a diagnostic score for the user based on the time series data. In implementations, the diagnostic score is calculated based on one or more of the following medical or physiological parameters: heart rate, ECG, blood pressure, blood sugar, body temperature, and respiratory rate or pattern, as well as on one or more impact parameters. In embodiments, the diagnostic score represents a likelihood of an LOC event, or the likelihood of a brain-related trauma (e.g., a mild TBI). In implementations, the monitoring device 404 calculates the diagnostic score within seconds (< 1 minute) of receiving the parameter data at step 402. In embodiments, the data analyzing module 411 of the monitoring device 404 implements step 505. In implementations, substeps 505A-505D are used to calculate the diagnostic score, as discussed below.

At substep 505A, the monitoring device 404 obtains a first set of values by assigning a first value (e.g., 0.75) to each medical or physiological parameter determined to exceed the predetermined threshold value at step 504 by less than standard deviation threshold value (e.g., less than two (2) standard deviations based on the baseline profile of the user). As an example, if a heart rate value of the user exceeds the predetermined threshold value at step 504, but does not exceed the threshold value by an amount that exceeds two standard deviations (e.g., where one standard deviation is 3), then the heart rate parameter would be assigned a value of 0.75.

At substep 505B, the monitoring device 404 obtains a second set of values by assigning a second higher value (e.g., 1.0) to each medical or physiological parameter to exceed the predetermined threshold value at step 504 by the standard deviation threshold value (e.g., more than two (2) standard deviations based on the baseline profile of the user) or higher. As an example, if a heart rate value of the user exceeds the predetermined threshold value at step 504 by an amount that exceeds the standard deviation threshold value of two standard deviations (wherein one standard deviation is 3), then the heart rate parameter would be assigned a value of 1.0.

At substep 505C, the monitoring device 404 obtains a third set of values by assigning another (third) score (e.g., 1.0) to one or more impact parameters determined to exceed one or more corresponding impact threshold values. In one example, the impact sensor is a blast sensor measuring overpressure. The term overpressure refers to the pressure caused by a shock wave over and above normal atmospheric pressure.

At substep 505D, the monitoring device 404 assigns a final score for a user by summing the first, second and third sets of values. In embodiments, one or more values are weighted based on the protective equipment profile of the user. For example, the first, second and/or third set of values may be weighted based on whether the level of protection of a user indicated in their protective equipment profile is high, medium, or low. In another example, the overall score may be weighted based on the level of protection of a user in a user’s protective equipment profile.

At step 506, the monitoring device 404 determines if the diagnostic score calculated at step 505 meets or exceeds a predetermined diagnostic threshold. In implementations, a diagnostic score meeting or exceeding a predetermined diagnostic threshold indicates an LOC and/or a TBI. It should be understood that calculating the diagnostic score and determining if the diagnostic score meets or exceeds a diagnostic threshold may be performed by the monitoring device 404 in real-time or near real-time. In implementations, steps 505 and 506 are performed by the monitoring device 404 based on real-time parameter data obtained at step 502, and steps 505 and 506 are performed in the order of seconds, rather than minutes (e.g., <60 seconds).

It can be appreciated that time is of the essence when detecting or diagnosing a possible LOC, concussion, or other brain injury, especially when the user is currently participating in an event that exposes them to further injury (e.g., a sporting event or field training). Implementations of the invention provide for real-time or near real-time diagnosis of users in situ, based on user profiles and sensor data (e.g., digital sensor data) obtained from wearable sensors of the user. For example, diagnosis of a potential LOC or TBI may occur at the site of a monitoring event (e.g., a sports stadium, a training field, etc.) in real-time, without the need to first manually evaluate the user. Complex diagnostic determinations of the invention would not be possible to implement by human calculation alone (i.e., within seconds of receiving the sensor data and based on user profile data). Moreover, human interpretation of digital sensor data (i.e., information represented as a string of discrete symbols each of which can take on one of only a finite number of values from some alphabet, such as letters or digits) in real-time could not reasonably be performed by manual methods alone. Additionally, embodiments of the invention provide for automated alerts to users and/or third parties based on real-time analysis of a user’s real-time sensor data.

At step 507, the monitoring device 404 automatically sends a notification or alert to one or more users or third parties based on the diagnosis score and/or one or more of the user parameters of the time series data meeting or exceeding a predetermined threshold value. In embodiments, the monitoring deice 404 automatically sends an alert to a participant device 406 of medical personnel (e.g., an emergency response team) when the diagnosis score meets or exceeds a predetermined diagnostic threshold.

In implementations, one or more participant devices 406 may be sent a wellness alert based on one or more of the user parameters of the time series data meeting or exceeding a predetermined threshold at step 504. For example, the monitoring device 404 may send an automatic alert to coaching staff that a player has an abnormally high body temperature. Thus, embodiments of the invention provide for the diagnosis of potential impact-related injury as well as wellness alerts regarding a variety of medical or physiological parameters. Additionally, implementations of the invention send notifications including instructions and/or diagnostic questions to guide a user in addressing a potential impact injury. In one example, if a final score is above a threshold of 5.5, the monitoring device 404 automatically sends an alert to a designated user that a screening and assessment for concussion and traumatic brain injury of the user by a medical professional is warranted. In one embodiment, an alert includes assessment questions that require answers from the user, such as through a user interface of a handheld participant device 406, wherein the alert is not closed or deactivated until the user has answered the questions. In such embodiments, the user’s answers to the questions may be forwarded to authorized users, such as authorized medical personnel.

Optionally, at step 508, the monitoring device 404 receives medical data for the user associated with the event at issue, and updates the stored data (e.g., time series data) in the data store 414 with the medical data. In one example, a user who was diagnosed with a possible LOC by the monitoring device 404 receives follow up medical data from an authorized user indicating a later medical diagnosis (e.g., a concussion) by medical personnel.

While above examples have been discussed with respect to a single user, it can be appreciated that, in implementations, the monitoring device 404 is configured to perform the above-identified functions for a plurality of users consecutively or simultaneously (e.g., for a team of players in a sporting event). Accordingly, the monitoring device 404 may accumulate data for a plurality of users over time.

At step 509, the monitoring device 404 automatically updates threshold values and/or weight values of the system (e.g., in the rules module 412) based on compiled data from multiple users in the data store 414, using ML tools. In implementations, the ML module 413 of the monitoring device 404 uses the user parameters, diagnosis scores, protective equipment profile and medical data of users as input data for ML algorithms, wherein the output of the ML algorithms is new threshold data for parameters and/or new weights to be applied based on protective equipment profiles. In embodiments, a classification algorithm is used to classify input data into predetermined categories, and a pattern recognition algorithm is utilized to identify patterns in the classified input data to determine thresholds that need to be adjusted up or down to more accurately predict an LOC, concussion or other TBI. In implementations, the ML module 413 is configured to determine what parameter or group of parameters have the greatest effect on LOC or TBI, and adjusts thresholds or weights accordingly in the rules module 412. Moreover, in embodiments, the ML module 413 is configured to determine how different protective equipment profiles impact user parameters and diagnostic scores, enabling adjustment of thresholds based on protective equipment of a user and providing insights into the effectiveness of different protective equipment and combinations of protective equipment.

Step 509 may be performed iteratively, over time, to develop the most accurate threshold and weight values for purposes of diagnosing potential LOC or TBIs. Steps 504 and 505 may rely on newly updated threshold values and/or weight values. Accordingly, embodiments of the invention constitute an improved diagnosing system that improves in accuracy over time based on compiled user data.

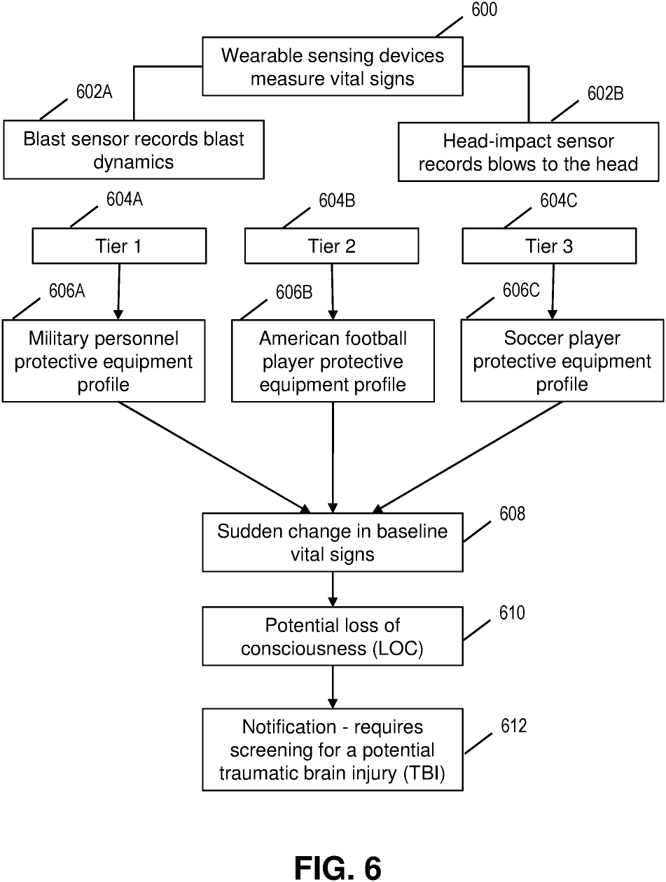

FIG. 6 is a diagram representing exemplary use scenarios with different tiers of user protection. Steps illustrated in FIG. 6 may be carried out in the environment of FIG. 4 and are described with reference to elements depicted in FIG. 4. In the example of FIG. 6, one or more wearable sensing devices (e.g., sensing devices 408) measure vital signs of users at 600. Different types of systems may be utilized for different types of users. For example, one or more impact sensors 420A may be in the form of one or more blast sensors recording blast dynamics, as indicated at 602A. Alternatively, one or more impact sensors 420A may be in the form of one or more head-impact sensors recording blows to the head, as indicated at 602B. In implementations, the monitoring device 404 recognizes different protective equipment profiles based on different protective gear or different combinations of protective gear. FIG. 6 shows a three-tier system, wherein Tier 1 (604A) represents the most protection, Tier 2 (604B) represents moderate protection, and Tier 3 (604C) represents limited protection of a user. In the example of FIG. 6, Tier 1 (604A) comprises a protective equipment profile 606A for military personnel, Tier 2 (604B) comprises a protective equipment profile 606B for an American football player, and Tier 3 (604C) comprises a protective equipment profile 606C for a soccer player.

In one example, the protective equipment profile 606A includes an integrated head protection system (IHPS), body armor including a modular scalable vest and a blast pelvic protector, eye protection, elbow and kneepads, and ear protection. In one example, the protective equipment profile 606B includes a helmet and shoulder pads, gloves, shoes, thigh pads, knee pads, mouthguard, and compression shorts. In some embodiments, the protective equipment profile 606B additionally includes neck rolls, elbow pads, hip pads, tail pads, and rib pads (e.g., made of synthetic materials including foam rubbers, elastics, and shock-resistant molded plastic). In one example, the protective equipment profile 606C includes a jersey, shorts, stockings, shin guards, and shoes. In some embodiments, the protective equipment profile 606C additionally includes a mouth guard, knee and elbow pads, gloves, and protective head gear.

With continued reference to FIG. 6, in implementations, the monitoring device 404 is configured to determine, for each tier 604A-604C, a sudden change in baseline vital signs of users at 608, and a potential LOC at 610. Additionally, embodiments of the monitoring device 404 enable alerts or notifications to be automatically sent to one or more users as indicated at 612, wherein the alerts or notifications indicate the requirement for a traumatic brain injury (TBI) screening, such as a concussion (e.g., mild TBI) screening.

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system/server 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system/server 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
receiving, by a computing device, real-time user parameter data from one or more sensors of the user during a monitoring event;
writing, by the computing device, the real-time user parameter data as time series data in a data store;
determining, by the computing device, that at least one parameter of the real-time user parameter data meets or exceeds a predetermined parameter threshold value;
calculating, by the computing device, a diagnostic score for the user based on the time series data, baseline parameter data of the user, and a determined protective equipment profile of the user; and
automatically diagnosing a potential traumatic brain injury (TBI) of the user in situ based on the diagnostic score meeting or exceeding a diagnostic threshold.

2. The method of claim 1, further comprising automatically sending, by the computing device, an alert to a remote participant device based on the diagnosing.

3. The method of claim 1, wherein the calculating the diagnostic score is performed by the computing device within a time frame of less than a minute from receiving the real-time user parameter data.

4. The method of claim 1, wherein the real-time user parameter data includes physiological parameter data and impact parameter data.

5. The method of claim 4, wherein the calculating the diagnostic score for the user comprises:
assigning a first value to each physiological parameter of the physiological parameter data that meets or exceeds a first threshold value based on the baseline parameter data of the user, thereby generating a set of first values;
assigning a second value higher than the first value to each physiological parameter that meets or exceeds a second higher threshold value based on the baseline parameter data of the user, thereby generating a set of second values; and
assigning a third value to one or more impact parameters of the impact parameter data that meets or exceeds another threshold value based on the determined protective equipment profile of the user, thereby generating a set of third values;
wherein the calculating the diagnostic score comprises summing the set of first values, the set of second values and the set of third values.

6. The method of claim 5, wherein the set of third values is generated by applying a weight value to the third value based on the determined protective equipment profile of the user.

7. The method of claim 1, wherein the determining that the at least one parameter of the real-time user parameter data meets or exceeds the predetermined parameter threshold value comprises determining that one or more impact parameters from an impact sensor meets or exceeds the predetermine parameter threshold value, wherein the calculating the diagnostic score is performed in response to the determining the one or more impact parameters meets or exceeds the predetermined parameter threshold value.

8. The method of claim 1, further comprising updating, by the computing device, the time series data with medical data related to the monitoring event.

9. The method of claim 8, further comprising updating, by a machine learning module of the computing device, one or more stored threshold rules based on compiled time series data from multiple users.

10. The method of claim 1, wherein the computing device includes software provided as a service in a cloud environment.

11. A computer program product comprising one or more computer readable storage media having program instructions collectively stored on the one or more computer readable storage media, the program instructions executable to cause a computing device to:
receive real-time user parameter data from one or more sensors of the user during a monitoring event, the real-time user parameter data including physiological parameter data and impact parameter data;
write the real-time user parameter data as time series data in a data store;
determine that at least one parameter of the real-time user parameter data meets or exceeds a predetermined parameter threshold value;
calculate a diagnostic score for the user based on the time series data, baseline parameter data of the user, and a determined protective equipment profile of the user; and
automatically diagnose a potential traumatic brain injury (TBI) of the user in situ based on the diagnostic score meeting or exceeding a diagnostic threshold.

12. The computer program product of claim 11, wherein the program instructions further cause the computing device to automatically send an alert to a remote participant device based on the diagnoses.

13. The computer program product of claim 11, wherein the calculating the diagnostic score is performed by the computing device within a time frame of less than a minute from receiving the real-time user parameter data.

14. The computer program product of claim 11, wherein the calculating the diagnostic score for the user comprises:
assigning a first value to each physiological parameter of the physiological parameter data that meets or exceeds a first threshold value based on the baseline parameter data of the user, thereby generating a set of first values;
assigning a second value higher than the first value to each physiological parameter that meets or exceeds a second higher threshold value based on the baseline parameter data of the user, thereby generating a set of second values; and
assigning a third value to one or more impact parameters of the impact parameter data that meets or exceeds another threshold value, wherein the third value is weighed based on the determined protective equipment profile of the user, thereby generating a set of third values;
wherein the calculating the diagnostic score comprises summing the set of first values, the set of second values and the set of third values.

15. The computer program product of claim 11, wherein the determining that the at least one parameter of the real-time user parameter data meets or exceeds the predetermined parameter threshold value comprises determining that one or more impact parameters from an impact sensor meets or exceeds the predetermine parameter threshold value, wherein the calculating the diagnostic score is performed in response to the determining the one or more impact parameters meets or exceeds the predetermined parameter threshold value.

16. The computer program product of claim 11, wherein the program instructions further cause the computing device to update, by a machine learning module of the computing device, one or more stored threshold rules based on compiled time series data from multiple users.

17. A system comprising:
a processor, a computer readable memory, one or more computer readable storage media, and program instructions collectively stored on the one or more computer readable storage media, the program instructions executable to cause a computing device to:
receive real-time user parameter data from one or more sensors of the user during a monitoring event, the real-time user parameter data including physiological parameter data and impact parameter data;
write the real-time user parameter data as time series data in a data store;
determine that at least one parameter of the real-time user parameter data meets or exceeds a predetermined parameter threshold value;
calculate a diagnostic score for the user based on the time series data, baseline parameter data of the user, and a determined protective equipment profile of the user;
automatically diagnose a potential traumatic brain injury (TBI) of the user in situ based on the diagnostic score meeting or exceeding a diagnostic threshold; and
send an alert based on the diagnosis to a remote participant device of a user.

18. The system of claim 17, wherein the calculating the diagnostic score is performed by the computing device within a time frame of less than a minute from receiving the real-time user parameter data.

19. The system of claim 17, wherein the calculating the diagnostic score for the user comprises:
assigning a first value to each physiological parameter of the physiological parameter data that meets or exceeds a first threshold value based on the baseline parameter data of the user, thereby generating a set of first values;
assigning a second value higher than the first value to each physiological parameter that meets or exceeds a second higher threshold value based on the baseline parameter data of the user, thereby generating a set of second values; and
assigning a third value to one or more impact parameters of the impact parameter data that meets or exceeds another threshold value, wherein the third value is weighed based on the determined protective equipment profile of the user, thereby generating a set of third values;
wherein the calculating the diagnostic score comprises summing the set of first values, the set of second values and the set of third values.

20. The system of claim 19, wherein the program instructions further cause the computing device to update, by a machine learning module of the computing device, one or more stored threshold rules based on compiled time series data from multiple users, wherein the time series data from multiple users is updated with medical data associated with monitoring events of the multiple users.
